(19) United States
(12) Patent Application Publication
(10) Pub. No.: US 2022/0015979 A1
(43) Pub. Date: Jan. 20, 2022

(54) Device for Supporting and Relieving the Arms of a User

(71) Applicant: Hilti Aktiengesellschaft, Schaan (LI)

(72) Inventors: Simon KAESMANN, Muenchen (DE); Lars MELZER, Hohenfurch (DE)

(21) Appl. No.: 17/414,830

(22) PCT Filed: Aug. 13, 2019
(86) PCT No.: PCT/EP2019/071717

§ 371 Date: Jun. 16, 2021

(30) Foreign Application Priority Data
Dec. 17, 2018 (EP) 18212835.5
Dec. 17, 2018 (EP) 18212840.5
Dec. 17, 2018 (EP) 18212922.1

(51) Int. Cl. A61H 1/02

(52) U.S. Cl. CPC A61H 1/0281; A61H 1/0277; A61H 2201/1676; A61H 2201/1673; A61H 2201/0192; A61H 2201/1638

(57) ABSTRACT

A device for supporting and relieving a load on an arm of a user includes an upper subregion, a lower subregion, and a joint that has a horizontal axis of rotation where the upper subregion and the lower subregion are connected by the joint such that the upper subregion is pivotable in an upper spatial direction and a lower spatial direction about the horizontal axis of rotation of the joint. The joint is pivotable in the upper spatial direction and the lower spatial direction in a first state and is pivotable only in the upper spatial direction in a second state.

### BACKGROUND AND SUMMARY OF THE INVENTION

The present invention relates to a device for supporting and relieving the load on a right arm and/or a left arm of a user, wherein the device comprises, for each side of the body, an upper subregion and a lower subregion, wherein the subregions can be connected by a joint such that the upper subregion is pivotable in an upper and a lower spatial direction about a horizontal axis of rotation of the joint. In particular, the supporting device is characterized in that it can be operated in preferably two different states.

Conventional supporting devices, which are also known for example as passive exoskeletons for supporting the upper extremities of a user, are known from the prior art. The upper extremities of a user are in particular the arms of the user. These passive supporting devices, in order to be able to ensure the support, frequently use the restoring force of resilient elements in order to compensate for example for the weight force of the upper extremities. In other words, the weight force can be absorbed by resilient elements, for example springs or spring mechanisms. In addition to the weight force of the arms, it is also possible for example to compensate for the additional weight force of a tool or some other object that the user holds in their hand.

However, the operating principle of the conventional supporting devices, which is based on absorption or compensation of forces by a resilient element, is associated with the drawback that the supporting force provided by the elastic element also continues to act in an end position, such that the user's arms are pushed out of a desired position. Furthermore, in the known supporting devices that are known from the prior art, the direction of the supporting force does not coincide, for many activities, with the actually required force direction that would be necessary to carry out a particular activity. Moreover, when the arms are lowered, the supporting force, which usually acts in the “upward” spatial direction, has to be overcome, and this is sometimes perceived as bothersome or challenging.

Therefore, the object of the present invention is to overcome the above-described drawbacks of conventional supporting devices and to provide a supporting device in which the supporting force does not continue to act beyond an end position, such that the arms of the user are not pushed out of a desired position. It would also be desirable, in the supporting device to be provided, for the direction of the supporting force to coincide with the direction of the force required for carrying out a particular activity. A further objective of the invention is for the bothersome overcoming of the preferably upwardly acting supporting or compensation force when lowering the arm no longer to be necessary.

The object is achieved in particular by a device for supporting and relieving the load on a right arm and a left arm of a user, wherein the device comprises, for each side of the body, an upper subregion and a lower subregion, wherein the upper subregion comprises an arm strut with an armrest, wherein the subregions of the supporting device are able to be connected by a joint that has a horizontal axis of rotation, such that the upper subregion is pivotable in an upper and a lower spatial direction about a horizontal axis of rotation of the joint. The device is characterized in that the joint is pivotable in the upper spatial direction and the lower spatial direction in a first state and is pivotable only in the upper spatial direction in a second state. In other words, the joint can be in a first state and in a second state, wherein the upper subregion of the supporting device is pivotable in the upper and the lower spatial direction in the first state and is pivotable only in the upper spatial direction in the second state. For the purposes of the invention, it is preferred for the changeover from the first state into the second, or vice versa, to be able to be effected by voice control, gesture control or by the use of electromyography (EMG). In a particularly preferred embodiment of the invention, a control device can be provided in the neck region of the user, preferably also being known as a “neck switch device”. For the purposes of the invention, it is preferred for the neck switch device to be controlled by means of a head movement of the user. In other words, the changeover from a first state into a second state of the joint of the supporting device can be effected by a movement of the user's head, wherein a corresponding control device can be arranged at the user's neck.

For the purposes of the invention, it is preferred for the device to be referred to as a supporting device or as an exoskeleton. For the purposes of the invention, the first state can preferably be referred to as a deactivated state and the second state of the joint as an activated state. Preferably, the upper subregion of the device is blocked in the lower spatial direction in the second state. The invention yields the advantage that the user maintains full freedom of movement in the upper and the lower spatial direction in the first state and the user's arm is supported in the second state in that the movement of the joint in the lower spatial direction is blocked. As a result, the arm strut of the upper subregion of the device cannot move downward and the user's (upper) arm is effectively supported downwardly.

For the purposes of the invention, it is preferred for the joint to be unblocked in both directions of rotation about a horizontal axis in the deactivated state. In other words, the joint is in a free-running state in both spatial directions. The directions of rotation are preferably also referred to as spatial direction or direction of movement for the purposes of the invention. In the context of the present invention, the two spatial directions are preferably the “upward” spatial direction and the “downward” spatial direction. The spatial directions can be defined with regard to a vertical imaginary axis, which can preferably be defined by the user's spine. The “upward” spatial direction corresponds preferably to a spatial direction along or parallel to this vertical imaginary axis, specifically in the direction of the user's head, i.e., in the direction of the highest point of the user. If the user is in a closed space, the “upward” spatial direction points preferably in the direction of the ceiling of the space. The “downward” spatial direction corresponds preferably to the spatial direction along or parallel to this vertical virtual axis, specifically in the direction of the user's feet, i.e., in the direction of the lowest point of the user. Preferably, the “downward” spatial direction points in the direction of a floor or ground on which the user of the supporting device is standing.

Since the joint is unblocked about the horizontal axis in both spatial directions or directions of movement in the deactivated state, the upper subregion of the supporting device can be moved in both spatial directions—upward and downward. This advantageously means that the user has unrestricted freedom of movement with regard to movements about the horizontal axis and is not restricted in terms of freedom of movement. In the activated state, the provided exoskeleton with the two operating states provides support for the user in the form of a bearing surface, in that the joint is blocked in one direction of movement. The blocked direction of movement is preferably the “downward” direction of movement. As a result of the preferred blocking of the “downward” direction of movement, support for the user's arm, which is preferably resting on the armrest, is advantageously achieved. In this way, the user can carry out for example overhead work, while the user can rest their arm or arms on the armrest of the arm strut and can carry out the overhead work from the resting position. In other words, the user of the device or the wearer of the exoskeleton can raise their arms further, wherein the arms are advantageously supported downwardly in the new position.

For the purposes of the invention, it is preferred for the upper subregion of the device to be present beneath the upper arm of a user and for the lower subregion to rest against the side of the user's torso. For each half of the body, the device has an upper and a lower subregion. Preferably, the device is provided to support both arms of a user. However, for the purposes of the invention, it may also be preferred for only one arm to be supported or relieved of load with the provided device during work. The upper subregion of the device comprises an arm strut with an armrest, on which the user's arm can rest. Preferably, the arm strut can comprise fastening means with which the arm can be fixed to the armrest of the arm strut. Preferably, the user's arms and the upper subregion of the supporting device can be moved jointly with one another. In other words, the movements of the upper subregions and of the arms can be coupled to one another. The lower subregion extends preferably along the side of the user's torso. For the purposes of the invention, it is preferred for the lower subregion to extend in particular along an upper region of the user's torso. This upper region can be located for example at the height of the user's chest. Preferably, the supporting device can comprise further fastening means for fastening the device to the user's torso.

For the purposes of the invention, it is preferred for the upper and the lower subregion of the supporting device to enclose an angle with one another, which is preferably an obtuse angle. The angle that can be enclosed by the subregions of the device can lie in a range between 0 and 180 degrees. For the purposes of the invention, it is preferred for the arms to be able to be freely moved substantially fully when using the supporting device. For example, the arms can hang down against the user's body or torso, this corresponding preferably to an angle of 0 degrees. The user's arms can also be stretched upward substantially parallel to the body, this corresponding preferably to an angle of 180 degrees. For the purposes of the invention, it is preferred for the angle values to be specified with reference to the user's spine, which extends preferably vertically and is present centrally in the user's body. For the purposes of the invention, it is very particularly preferred for the joint of the supporting device to be blocked in the entire range of movement of the user, wherein the range of movement covers preferably an angular range of 0 to 180 degrees.

For the purposes of the invention, it is preferred for the horizontal axis of rotation of the joint to be present in the vicinity of the crook of the arm or in the region of the user's armpit. The joint can also be provided at other locations, however, for example in the region of the user's shoulder or next to the user's shoulder or above the user's shoulder. In other words, the upper and the lower region of the supporting device can be extended along a side of the user's body or torso or be arranged in the vicinity of the flank of the body. Preferably, the upper subregion of the supporting device extends on an underside of the upper arm of the user, which preferably lies opposite the side of the user's torso. Preferably, the lower region of the supporting device is arranged against this side of the user's torso at chest height.

In one embodiment of the invention, it is preferred for the joint to be designed to switch between free-running and a blocked spatial direction. These directions of movement are referred to preferably as “allowed direction of rotation” and as “blocked direction of rotation” for the purposes of the invention. It is very particularly preferred for the joint to comprise a ratchet, wherein the joint with the ratchet is designed to switch between free-running and a blocked spatial direction. For the purposes of the invention, it is very particularly preferred to use a fine tooth ratchet.

Preferably, the joint comprises an adapted ratchet, wherein the ratchet is designed to allow the switching between free-running and a blocked spatial direction. The ratchet used in the context of the present invention is preferably adapted such that, rather than it being possible—as in a conventional ratchet—to choose between two free directions of rotation, it is possible to switch between free-running and a blocked direction of rotation. In this regard, the operating principle of the provided supporting device differs from conventional passive exoskeletons in that use is not made of a spring mechanism in order to compensate for any forces, for example the weight force of an arm, but rather that preferably a supporting capacity for the user's arm is created in that, in the context of the present invention, a joint that blocks in one spatial direction is used. Thus, in the deactivated state of the supporting device, the user can move their arms upward at all times with full freedom of movement. By contrast, in the activated state of the supporting device, the user can move their arms further upward, but can no longer move them downward in the blocked direction of movement. In the activated state, the device, or the upper subregion thereof, remain advantageously in a middle rest position, in which the user's arm is supported by the upper subregion of the device. For the purposes of the invention, it is preferred, in the context of the provided supporting device, for a joint to be used that comprises preferably an adapted ratchet. While it is possible, in a conventional ratchet, to choose to between two directions of rotation, in the adapted ratchet it is preferred for the joint of the provided exoskeleton to be able to be switched between free-running and a blocked direction. Free-running corresponds preferably to the deactivated state. For the purposes of the invention, it is preferred for the joint, in the activated state, to absorb forces in the one direction, while it slips through with minimum forces in the other direction. In the deactivated state, which, for the purposes of the invention, is preferably also referred to as the free-running state, no teeth are engaged within the ratchet, and so the joint slips through in both directions of rotation in the deactivated state.

For the purposes of the invention, it is preferred for the upper subregion to be able to be fastened to the lower subregion via a coupling rod with a partial gear. This connecting mechanism, which preferably comprises at least the coupling rod and the partial gear, is designed to compensate for shoulder movements of the user. It is possible, when the user lifts their arms, for the user's shoulder, or their shoulder joints, also to be raised. This raising of the shoulders or of the shoulder joints occurs in particular once the arms have been raised by more than 90 degrees, i.e., when parts of the user's arms are located above the shoulder girdle. Preferably, the connecting mechanism ensures that the supporting device follows the shoulder or the shoulder movement, such that the axes of rotation of the device and of the shoulder joint advantageously continue to coincide.

The shoulder movements of the user can consist for example in that the shoulders are raised while a user is working. For the purposes of the invention, this raising of the shoulder is preferably also referred to as shoulder elevation. Preferably, the supporting device is designed to synchronize an elevation of a user's shoulders with the device. In particular, the structure of the provided supporting device makes it possible for the rotary joint of the device, when the upper extremity is raised, to be adjusted upward by a height difference Ah. This has the advantageous result that a height offset is compensated. As a result of this compensation of the shoulder elevation, the supporting device can follow movements of the human body better, and so much more effective support of the user is allowed than is the case in conventional systems without elevation compensation.

For the purposes of the invention, it is preferred for the elevation of the shoulder(s) to start from a particular angular position of the upper extremities, i.e., of the arms. Preferably, in this angular position, a vertically upright component of the supporting device is lengthened such that the joint of the supporting device follows the shoulder joint. This is brought about preferably by the provision of a mechanism that is coupled to the angular position of the arm and can comprise for example at least one coupling rod and a partial gear. As a result, the natural combination of movements for the upper extremities is advantageously retained and the attachment points between the human body and the supporting device are maintained.

For the purposes of the invention, it is preferred for a rack to be guided within a sleeve that is rotatable about a vertical axis. The rack can be moved via a partial gear, such that the sleeve and the attached ratchet are displaced in the direction of the plain bearing. This displacement takes place preferably in the “upward” spatial direction. The partial gear is moved preferably by a mechanical coupling to the movement of the armrest of the supporting device. For example, the coupling rod can comprise an opening, which can be in the form for example of a slot. The provision of the opening allows the arm strut to be able to be lowered without a force needing to be exerted on the partial gear. Preferably, a lever of the partial gear can be provided at one end of the opening, with the result that further raising of the armrest via the partial gear causes displacement of the rack. The joint can preferably be guided upward by a spindle or an actuator. As an alternative to the mechanical coupling, the angular position of the arm strut can be determined via a suitable sensor system, in order to compensate for an elevation of the shoulders of a user of the supporting device.

The coupling rod extends preferably substantially parallel to the lower subregion of the supporting device, while an angle that the coupling rod encloses with the upper subregion can vary depending on the state in which the supporting device is operated or depending on the angle that the upper and the lower subregion enclose with one another. The coupling rod is preferably connected to the partial gear. For the purposes of the invention, it is particularly preferred for the coupling rod to be connected to the lower subregion of the supporting device via the partial gear. The lower subregion comprises preferably an attachment plate, and a rack, which are formed substantially parallel to one another. The rack can comprise a sleeve, wherein plain bearings are provided on the rack above and below the sleeve. In one embodiment of the invention, it is preferred for the lower subregion of the supporting device to comprise at least one plain bearing in order to ensure vertical adjustability of the upper subregion. Preferably, the number of plain bearings can be varied as required in the context of the present invention.

For the purposes of the invention, it is preferred for the provided supporting device to be able in particular to relieve the user of load during overhead work. It can in particular result in load relief on the upper extremities during activities above shoulder level, wherein in particular a load-relief capacity for the user is created by the provision of a bearing surface for the upper arms. In the deactivated state, this bearing surface follows the movements of the upper extremities without providing support. In the activated state, the provided supporting device supports the upper arm from below, while it continues to follow upward movements in order, in the new position, to always provide support in the opposite direction again.

Preferably, the provided supporting device can comprise means for activating the supporting device. The activation can be affected for example by the adoption of a head position that is typical for overhead work by the user. The adoption of this head position can be used by the activation means to activate the functions of the supporting device. It was completely surprising that, with the activation means, a capacity can be created to control the provided supporting device in that individual functions of the supporting device can be activated or deactivated. In this regard, the activation means can also be referred to as a neck switch device of the supporting device. In order that the user can focus on an overhead place of work, an inclination of the head toward the rear usually occurs, wherein the wording “toward the rear” is understood preferably, for the purposes of the invention, as “into the neck”. In other words, many users intuitively tip their head back into the neck during overhead work, in order to be able to optimally see their place of work for the hands. This movement of the head into the neck can advantageously be used to activate individual functions of the supporting device or to control the supporting device. In particular, the invention allows the head movement to be translated into the activation of the joint of the supporting device. In particular, the activation can represent mechanical activation using the neck switch. For example, via a two-part lever construction, a Bowden cable can be moved, which switches the joint of the supporting device. Likewise, for the purposes of the invention, it may be preferred for an inclination of the head to be determined by a position sensor that is integrated for example in a helmet or goggles of the user of the supporting device. As a result of the provision of suitable actuator technology, the joint of the supporting device can be switched by a movement of the user's head, for example into their neck.

Further advantages will become apparent from the following description of the figures. The figures, the description and the claims contain numerous features in combination. A person skilled in the art will expediently also consider the features individually and combine them to form useful further combinations.

In the figures, identical and similar components are denoted by the same reference signs.

### BRIEF DESCRIPTION OF THE DRAWINGS

FIG. 1 shows a schematic illustration of a preferred configuration of the invention, in particular an illustration of the deactivated and the activated state;

FIG. 2 shows a schematic side view of a preferred configuration of the supporting device;

FIG. 3 shows a detail illustration of a preferred embodiment of the ratchet;

and

FIG. 4 shows a further schematic side view of a preferred configuration of the supporting device.

### DETAILED DESCRIPTION OF THE DRAWINGS

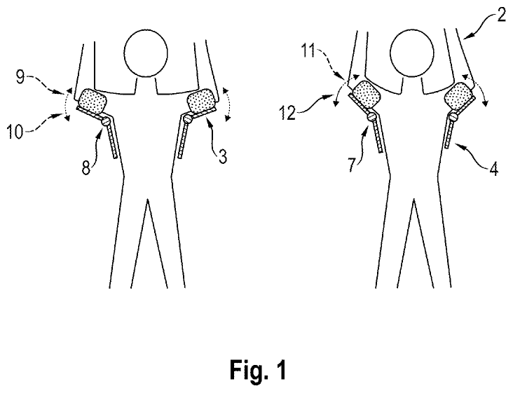

FIG. 1 shows, in the left-hand half of the image, the deactivated state and, in the right-hand half of the image, the activated state of a preferred configuration of the provided supporting device (1). Illustrated in each case is a user, who wishes to carry out for example overhead work with a power tool using their arms (2). Shown in the schematic illustration in FIG. 1 is in particular the upper subregion (3) and the lower subregion (4) of the supporting device (1). The upper subregion (3) is present preferably on an inner side of the user's upper arm (2), wherein the expression “inner side” preferably relates to the fact that, with the arms (2) not raised, one side of the arm faces the torso. This side of the arm (2) facing the torso is preferably referred to, for the purposes of the invention, as the inner side, while the side of the torso facing the upper arm is referred to as the side of the torso. Preferably, the lower subregion (4) of the supporting device (1) is present against this side of the user's torso. In other words, the lower subregion of the supporting device (1) is designed to be worn by a user in the region of the torso, in particular at chest height.

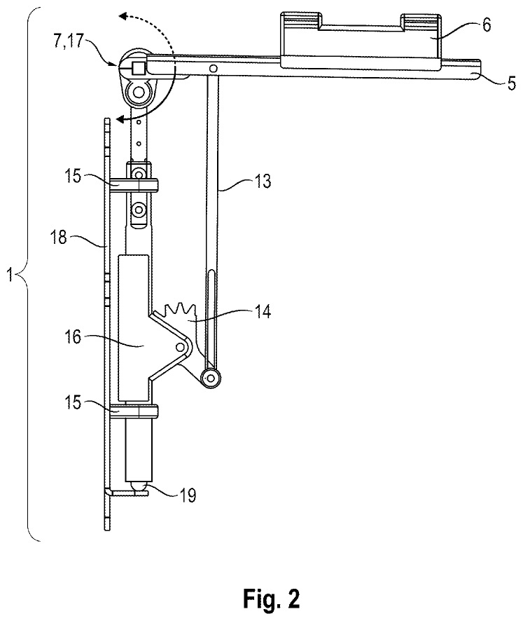

The upper region (3) of the supporting device (1) comprises preferably an arm strut (5) and an armrest (6) (illustrated in FIG. 2). When the supporting device (1) is used, the user's arms (2) can rest on the armrests (6). The upper subregion (3) and the lower subregion (4) of the supporting device (1) are preferably connected together via a joint (7). The joint (7) is arranged preferably in an axis of rotation (8), allowing horizontal pivotability of the upper subregion (3) with respect to the lower subregion (4) of the supporting device (1). Preferably, a horizontal axis of rotation extends through a pivot point (8) or the pivot point (8) is preferably part of this horizontal axis of rotation. The pivotability in particular of the upper subregion (3) of the supporting device (1) preferably has the effect that in particular the upper subregion (3) can move in an upper spatial direction (9) and a lower spatial direction (10). For the purposes of the invention, it is preferred for the upper subregion (3) of the supporting device (1) to be able to rotate about the horizontal axis (8) or the corresponding pivot point (8). For the purposes of the invention, it may furthermore be preferred for the upper subregion (3) and the lower subregion (4) of the supporting device (1) to additionally be able to rotate about a vertical axis, which extends preferably substantially parallel to the longitudinal axis of the body.

The pivotability is indicated in FIG. 1 in particular by the arrows in the region of the user's arms (2). In this case, the upward arrow stands for an upward movement of the upper subregion (3) of the supporting device (1) and the downward arrow stands for a downward movement of the upper subregion (3) of the supporting device (1). In the deactivated state of the exoskeleton (1), both directions of movement (9, 10) are allowed by the joint (7), i.e., both spatial directions (9, 10) are allowed directions of rotation (11) in the deactivated state.

In the right-hand half of the image in FIG. 1, the activated state of the supporting device (1) is shown. In the activated state, an upward movement of the user's arms (2), i.e., in the upper spatial direction (9), continues to be possible. Thus, the upper direction of rotation (9) of the upper subregion (3) of the supporting device (1) represents an allowed direction of rotation (11) in the activated state. The joint (7) blocks in a lower direction of rotation (10) in the activated state. Therefore, in the activated state, the lower direction of rotation (10) represents a blocked direction of rotation (12). The allowed direction of rotation (11) and the blocked direction of rotation (12) are indicated by arrows in FIG. 1.

FIG. 2 shows a schematic side view of a preferred configuration of the supporting device (1). In particular the structure of the subregion (3, 4) of the supporting device (1) is illustrated. The arm strut (5) and the armrest (6) are illustrated, which form the upper subregion (3) of the supporting device (1). Also visible are the further constituents of the lower subregion (4) and of the joint (7), which connects the subregions (3, 4) together. The joint (7) can comprise in particular a ratchet (17), which can preferably be in the form of a fine tooth ratchet. The lower subregion (4) comprises in particular a rack (19), which extends preferably substantially parallel to an attachment plate (18) and a coupling rod (13). The coupling rod (13) is preferably connected to the arm strut (5) of the upper subregion (3) of the supporting device (1). The attachment plate (18) can preferably be connected to the rack (19) by plain bearings (15). Furthermore, a sleeve (16) is arranged on the rack (19), the sleeve (16) being able to be connected to a partial gear (14).

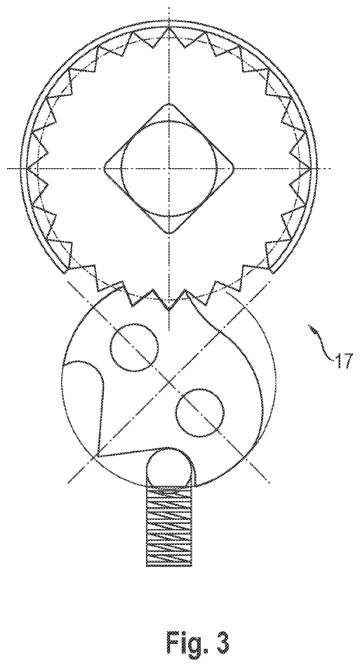

FIG. 3 shows a detail illustration of a preferred embodiment of the ratchet. In the state illustrated in FIG. 3, the joint (7) or the ratchet is in the activated state.

For the purposes of the invention, it is preferred for the joint (7) or the ratchet (17), in the activated state, to absorb forces in the one direction, while it slips through with minimum forces in the other direction. The force absorption in the one direction corresponds preferably to the blocking of the corresponding spatial direction. In particular, the joint (7) blocks the downward direction of rotation (10) in the activated state, so as to prevent the upper subregion (3) of the supporting device (1) from moving downward in the activated state. As a result, the lower direction of rotation or of movement (10) corresponds to the blocked direction of rotation (12) in the activated state of the supporting device (1). The slipping through of the joint (7) corresponds preferably to the free or allowed direction of movement (11). In the deactivated state, both the upper direction of rotation (9) and the lower direction of rotation (10) are allowed directions of rotation (11). In particular, in the deactivated state, no teeth are engaged within the ratchet (17), and so the joint (7) can slip through in both directions of rotation (9, 10) in the deactivated state. The allowed directions of rotation (11) are preferably also referred to as free-running for the purposes of the invention.

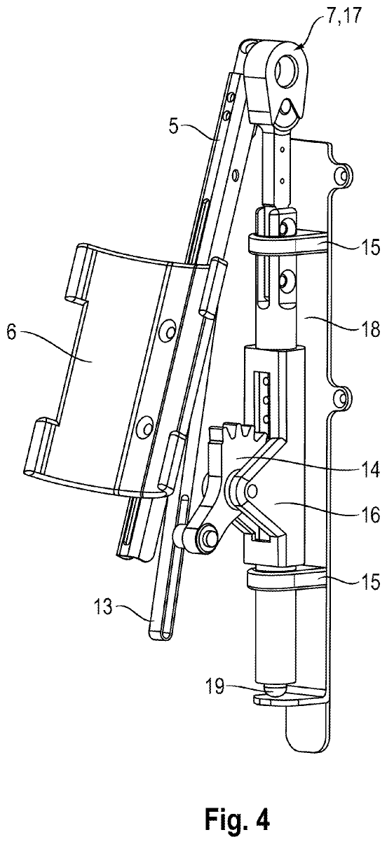

FIG. 4 shows a further schematic side view of a preferred configuration of the supporting device (1).

### LIST OF REFERENCE CHARACTERS:

1 Device/supporting device
2 Arms of a user
3 Upper subregion
4 Lower subregion
5 Arm strut
6 Armrest
7 Joint
8 Horizontal pivot point or horizontal axis of rotation
9 Upper spatial direction
10 Lower spatial direction
11 Allowed direction of rotation
12 Blocked direction of rotation
13 Coupling rod
14 Partial gear
15 Plain bearing
16 Sleeve
17 Ratchet
18 Attachment plate
19 Rack

1.-6. (canceled)

7. A device for supporting and relieving a load on an arm of a user, comprising:
an upper subregion, wherein the upper subregion includes an arm strut and an armrest;
a lower subregion; and
a joint that has a horizontal axis of rotation, wherein the upper subregion and the lower subregion are connected by the joint such that the upper subregion is pivotable in an upper spatial direction and a lower spatial direction about the horizontal axis of rotation of the joint;
wherein the joint is pivotable in the upper spatial direction and the lower spatial direction in a first state and is pivotable only in the upper spatial direction in a second state.

8. The device according to claim 7, wherein the upper subregion is disposable beneath an upper part of the arm of the user and the lower subregion is restable against a side of a torso of the user.

9. The device according to claim 7, wherein the horizontal axis of rotation is disposable in a vicinity of a crook of the arm of the user or in a region of an armpit of the user.

10. The device according to claim 7, wherein the joint is configured to switch between the first state and the second state.

11. The device according to claim 7, wherein the upper subregion is fastened to the lower subregion via a coupling rod with a partial gear.

12. The device according to claim 7, wherein the lower subregion includes a plain bearing for vertical adjustability of the upper subregion.
